(19) United States
(12) Patent Application Publication
(10) Pub. No.: US 2007/0155999 A1
(43) Pub. Date: Jul. 5, 2007

(54) Olefin production via oxygenate conversion

(76) Inventors: Peter R. Pujado, Kildeer, IL (US); Bipin V. Vora, Naperville, IL (US); John J. Senetar, Naperville, IL (US); Lawrence W. Miller, Palatine, IL (US)

Correspondence Address: HONEYWELL INTELLECTUAL PROPERTY INC;PATENT SERVICES, 101 COLUMBIA DRIVE, P O BOX 2245 MAIL STOP AB/2B, MORRISTOWN, NJ, 07962, US

(21) Appl. No.: 11/322,412

(22) Filed: Dec. 30, 2005

(51) Int. Cl. C07C 1/00; B01J 8/00

(52) U.S. Cl. 585/327; 585/638; 422/190

(57) ABSTRACT

Improved processing for the production of light olefins via oxygenate conversion processing is provided. Synthesis gas conversion such as to produce an effluent including at least methanol can be integrated with oxygenate conversion processing such as to produce an oxygenate conversion reactor effluent including at least light olefins and dimethyl ether. At least a portion of the oxygenate conversion reactor effluent can be contacted with such produced methanol to effect recovery of dimethyl ether from the oxygenate conversion reactor effluent.

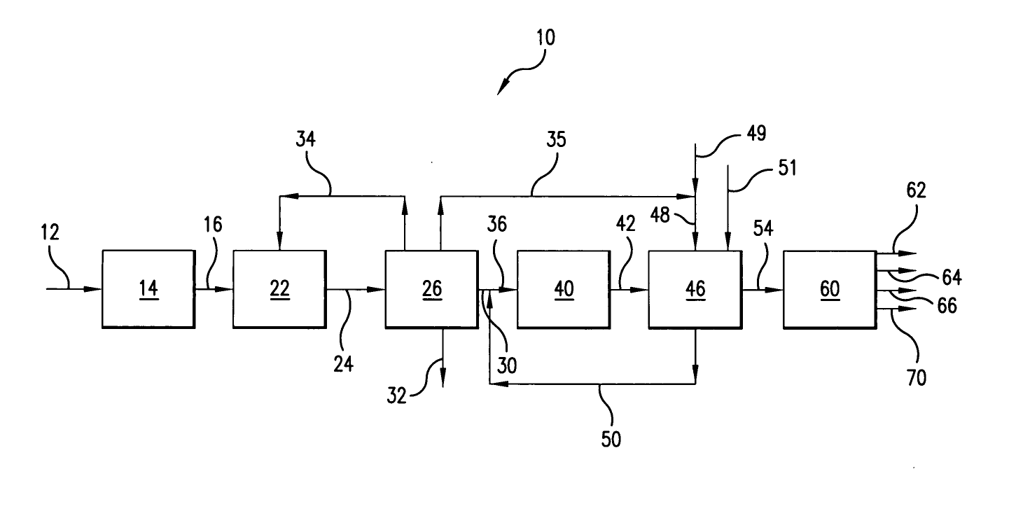

### BACKGROUND OF THE INVENTION

This invention relates generally to the production of olefins and, more specifically, to the production of olefins, particularly light olefins, via oxygenate conversion processing.

A major portion of the worldwide petrochemical industry is involved with the production of light olefin materials and their subsequent use in the production of numerous important chemical products. Such production and use of light olefin materials may involve various well-known chemical reactions including, for example, polymerization, oligomerization, alkylation reactions. Light olefins generally include ethylene, propylene and mixtures thereof. These light olefins are essential building blocks used in the modem petrochemical and chemical industries. A major source for light olefins in present day refining is the steam cracking of petroleum feeds. For various reasons including geographical, economic, political and diminished supply considerations, the art has long sought sources other than petroleum for the massive quantities of raw materials that are needed to supply the demand for these light olefin materials.

The search for alternative materials for light olefin production has led to the use of oxygenates such as alcohols and, more particularly, to the use of methanol, ethanol, and higher alcohols or their derivatives or other oxygenates such as dimethyl ether, diethyl ether, etc., for example. Molecular sieves such as microporous crystalline zeolite and non-zeolitic catalysts, particularly silicoaluminophosphates (SAPO), are known to promote the conversion of oxygenates to hydrocarbon mixtures, particularly hydrocarbon mixtures composed largely of light olefins.

Such processing, wherein the oxygenate-containing feed is primarily methanol or a methanol-water combination (including crude methanol), typically results in the release of significant quantities of water upon the sought conversion of such feeds to light olefins. For example, such processing normally involves the release of about 2 mols of water per mol of ethylene formed and the release of about 3 mols of water per mol of propylene formed. The presence of such increased relative amounts of water can significantly increase the potential for hydrothermal damage to the oxygenate conversion catalyst. Moreover, the presence of such increased relative amounts of water significantly increases the volumetric flow rate of the reactor effluent, resulting in the need for larger sized vessels and associated processing and operating equipment.

U.S. Pat. No. 5,714,662 to Vora et al., the disclosure of which is hereby incorporated by reference in its entirety, discloses a process for the production of light olefins from a hydrocarbon gas stream by a combination of reforming, oxygenate production, and oxygenate conversion wherein a crude methanol stream (produced in the production of oxygenates and comprising methanol, light ends, and heavier alcohols) is passed directly to an oxygenate conversion zone for the production of light olefins.

While such processing has proven to be effective for olefin production, further improvements have been desired and sought. For example, there is an ongoing desire and need for reducing the size and consequently the cost of required reaction vessels. Further, there is an ongoing desire and need for processing schemes and arrangements that can more readily handle and manage the heat of reaction and by-product water associated with such processing.

### SUMMARY OF THE INVENTION

A general object of the invention is to provide improved processing schemes and arrangements for the production of olefins, particularly light olefins.

A more specific objective of the invention is to overcome one or more of the problems described above.

The general object of the invention can be attained, at least in part, through specified methods for producing light olefins. In accordance with one embodiment, there is provided an integrated process for oxygenate synthesis and conversion to light olefins. More specifically, such a process involves contacting a synthesis gas-containing feedstock in a synthesis gas conversion reactor zone with a catalyst material and at reaction conditions effective to produce a synthesis gas conversion reactor section effluent comprising at least methanol. The process also involves contacting an oxygenate-containing feedstock comprising at least one oxygenate-containing feedstock material selected from the group consisting of methanol and dimethyl ether in an oxygenate conversion reactor zone with an oxygenate conversion catalyst and at reaction conditions effective to convert the oxygenate-containing feedstock to produce an oxygenate conversion reactor zone effluent comprising light olefins and by-product dimethyl ether. At least a portion of the oxygenate conversion reactor zone effluent is contacted with at least a portion of the synthesis gas conversion reactor zone effluent methanol effective to recover by-product dimethyl ether from the oxygenate conversion reactor zone effluent.

The prior art generally fails to provide processing schemes and arrangements for the production of olefins and, more particularly, to the production of light olefins from an oxygenate-containing feed and which processing schemes and arrangements are as simple, effective and/or efficient as may be desired.

An integrated process for oxygenate synthesis and conversion to light olefins, in accordance with another embodiment, involves contacting a synthesis gas-containing feedstock in a synthesis gas conversion reactor zone with a catalyst material and at reaction conditions effective to produce a synthesis gas conversion reactor zone effluent comprising product dimethyl ether, other synthesis gas conversion products, including methanol and water, and unreacted synthesis gas. Unreacted synthesis gas is desirably separated from the product dimethyl ether and the other synthesis gas conversion products. The separated unreacted synthesis gas can then be recycled to the synthesis gas conversion reactor zone for contact with the catalyst material at reaction conditions effective to produce additional synthesis gas conversion reactor zone effluent. At least a portion of the other synthesis gas conversion product methanol is desirably separated from the product dimethyl ether and from the other synthesis gas conversion product water. The process also involves contacting an oxygenate-containing feedstock comprising methanol and dimethyl ether in an oxygenate conversion reactor zone with an oxygenate conversion catalyst and at reaction conditions effective to convert the oxygenate-containing feedstock to produce an oxygenate conversion reactor zone effluent comprising light olefins and by-product dimethyl ether. At least a portion of the oxygenate conversion reactor zone effluent is contacted with at least a portion of the separated other synthesis gas conversion reactor zone effluent methanol effective to recover by-product dimethyl ether from the oxygenate reactor zone effluent. The process further involves recycling the recovered by-product dimethyl ether to the oxygenate conversion reactor zone for contact with the oxygenate conversion catalyst at reaction conditions effective to convert the oxygenate-containing feedstock to produce additional oxygenate conversion reactor zone effluent.

There is also provided an integrated system for oxygenate synthesis and conversion to light olefins. In accordance with one preferred embodiment, such as system includes a synthesis gas conversion reactor zone for contacting a synthesis gas-containing feedstock with a synthesis gas conversion catalyst and at reaction conditions effective to convert the synthesis gas-containing feedstock to produce a synthesis gas conversion reactor zone effluent comprising product dimethyl ether, other synthesis gas conversion products such as methanol and water, and unreacted synthesis gas. A separation zone also is provided. The separation zone is effective for separating the synthesis gas conversion reactor zone effluent to form a recycle stream of unconverted synthesis gas, a first process stream comprising methanol and an oxygenate-containing feed stream comprising at least one oxygenate-containing material selected from the group consisting of methanol and dimethyl ether. An oxygenate conversion reactor zone is provided for contacting an oxygenate-containing feedstock comprising at least one oxygenate-containing feedstock material selected from the group consisting of methanol and dimethyl ether with an oxygenate conversion catalyst and at reaction conditions effective to convert the oxygenate-containing feedstock to produce an oxygenate conversion reactor zone effluent comprising light olefins and by-product dimethyl ether. The system further includes a separation system effective to separate by-product dimethyl ether from the oxygenate conversion reactor zone effluent via methanol absorption of such by-product dimethyl ether.

As used herein, references to “light olefins” are to be understood to generally refer to C₂ and C₃ olefins, i.e., ethylene and propylene.

The term “carbon oxide” refers to carbon dioxide and/or carbon monoxide.

The term “synthesis gas”, also sometimes referred to as “syn gas”, generally refers to a combination of hydrogen and carbon oxides such as produced by or in a synthesis gas production facility from a hydrocarbon gas such as derived from natural gas or from the partial oxidation of a petroleum or coal residue. Normally, synthesis gas is identified as a combination of H₂ and CO at various ratios, sometimes with minor amounts of CO₂.

The term “by-product dimethyl ether” generally refers to dimethyl ether such as may remain unreacted after a reaction or as may be formed through a side or minor concurrent reaction.

Other objects and advantages will be apparent to those skilled in the art from the following detailed description taken in conjunction with the appended claims and drawing.

### BRIEF DESCRIPTION OF THE DRAWING

The FIGURE is a simplified schematic diagram of a process for the production of olefins and, more specifically, a process for the production of olefins, particularly light olefins, via oxygenate conversion processing.

### DETAILED DESCRIPTION OF THE INVENTION

Referring to the FIGURE, there is illustrated a simplified schematic process flow diagram for a process scheme, generally designated by the reference numeral 10, for the production of olefins, particularly light olefins, via oxygenate conversion processing. It is to be understood that no unnecessary limitation to the scope of the claims which follow is intended by the following description. Those skilled in the art and guided by the teachings herein provided will recognize and appreciate that the illustrated process flow diagram has been simplified by the elimination of various usual or customary pieces of process equipment including some heat exchangers, process control systems, pumps, fractionation systems, and the like. It may also be discerned that the process flow depicted in the FIGURE may be modified in many aspects without departing from the basic overall concept of the invention.

A hydrocarbon feed stream, such as in gaseous form and designated by the reference numeral 12, is passed to a synthesis gas generation or production zone 14 to produce a synthesis gas-containing stream 16. As will be appreciated by those skilled in the art and guided by the teachings herein provided, a wide variety of suitable or appropriate hydrocarbon feed streams can be used in the practice of such embodiment. For example, a suitable hydrocarbon feed stream may desirably comprise a natural or synthetic natural gas stream such as produced from a natural gas, coal, shale oil, residua or combination thereof and such as typically comprises methane and ethane and such as can be processed in a synthesis gas production facility to remove impurities such as sulfur compounds, nitrogen compounds, particulate matter, and condensibles and to provide a synthesis gas stream reduced in contaminants and containing hydrogen and carbon oxide in a desired molar ratio. Thus, it is to be understood that the broader practice of the invention is not necessarily limited by or to the use of particular or specific hydrocarbon feed streams.

The synthesis gas generation or production zone 14, or synthesis gas production facility, can operate at conventional operating conditions such as at a reaction temperature ranging from about 800° to about 950° C., a pressure ranging from about 10 to about 30 bar, and a water to carbon molar ratio ranging from about 2.0 to about 3.5. In the synthesis gas generation zone 14, impurities such as sulfur compounds, nitrogen compounds, particulate matter, and condensibles are desirably removed such as in a conventional manner to provide the synthesis gas-containing stream 16 that is reduced in contaminants and containing a molar ratio of hydrogen to carbon oxide (carbon monoxide plus carbon dioxide) ranging from about 2 to about 3, and more typically the molar ratio of hydrogen to carbon oxide varies from about 2.0 to about 2.3. Optionally (not shown), this ratio may be varied according to the shift reaction (1), shown below, over a copper/zinc or chromium oxide catalyst such as in a conventional manner:
CO+H₂O→CO₂+H₂ (1)

Those skilled in the art and guided by the teachings herein provided will appreciate that such processing generally corresponds to a steam reforming operation such as practiced for the production of synthesis gas from natural gas and other light hydrocarbons. However, as indicated above, synthesis gas can be produced from various hydrocarbons. For heavy hydrocarbons, catalytic steam reforming is generally not practical. When processing such materials, noncatalytic partial oxidation or gasification is more commonly used. Such processing typically involves the injection of oxygen (and optionally some steam) at temperatures as high as 1300° C. and pressures up to about 100 bar. For light, clean feeds partial oxidation can also be used in addition to steam reforming—various combinations exist in the form of autothermal reformers, gas-heated reformers, and the like. Because such units can be more compact, such partial oxidation approaches are generally favored in modem synthesis gas units, such as for the production of methanol at capacities in excess of about 4,000 MT/day methanol, for example. Steam reformer units are usually limited to a maximum of about 3,500 MT/day methanol.

Whether using steam reforming or some form of partial oxidation (e.g., autothermal reactors), catalytic processes are usually limited to clean (hydrotreated feeds) like natural gas or light hydrocarbons. Heavy feeds like refinery residues and coal are too dirty (e.g., contain high levels of contaminants) for effective hydrotreating—in such cases noncatalytic partial oxidation (or gasification) may be used, with the contaminants being removed from the effluent synthesis gas.

Returning to the FIGURE, the synthesis gas-containing stream 16 is passed to a synthesis gas conversion reactor zone 22. In the synthesis gas conversion reactor zone 22, at least a portion of the synthesis gas will undergo conversion to form reduction products of carbon oxides, such as alcohols, such as methanol and/or their derivatives, or other oxygenates such as dimethyl ether, diethyl ether, etc., for example. More specifically, such conversions can generally occur at conditions including a reactor temperature in the range of about 150° C. (300° F.) to about 450° C. (850° F.) at a pressure typically in the range of about 1 to about 1000 atmospheres over a variety of catalysts.

The methanol synthesis reaction can benefit from the coproduction of dimethyl ether. In particular, methanol synthesis from hydrogen gas (H₂) and carbon monoxide (CO) is generally equilibrium limited with typical per-pass conversion rates in the range of about 25% to about 30% at a pressure of 50 to 100 bar and a temperature in the range of about 250° to about 300° C. However, if methanol is converted to dimethyl ether, either while the methanol is being produced or shortly thereafter, the equilibrium can desirably be shifted to more favorable, higher synthesis gas conversions. As a result of such increased synthesis gas conversion rates, the amount or extent of recycle of unreacted synthesis gas, as more fully described below, can be decreased or minimized.

For example, methanol can be produced by passing synthesis gas over a supported mixed metal oxide catalyst of CuO and ZnO. Methanol conversion to dimethyl ether can be accomplished by passing such methanol over an acidic catalyst such as comprising gamma-alumina or the like. Both of the methanol formation and the methanol conversion to dimethyl ether reactions are exothermic and typically best operate at a temperature in the range of about 250° to about 300° C.

In accordance with certain preferred embodiments, the conversion of methanol to dimethyl ether can be accomplished by passing such methanol over an acidic catalyst such as comprising gamma-alumina or the like. Both of the methanol formation and the methanol conversion to dimethyl ether reactions are exothermic and typically best operate at a temperature in the range of about 250° to about 300° C.

In accordance with certain preferred embodiments, the conversion of methanol to dimethyl ether can be accomplished by using a mixed catalyst system in the reactor used for methanol synthesis. In accordance with certain alternative preferred embodiments, the conversion of methanol to dimethyl ether can be accomplished by employing a reactor with alternating beds of methanol synthesis catalyst and methanol-to-dimethyl ether conversion catalyst. In accordance with certain yet other alternative preferred embodiments, the conversion of methanol to dimethyl ether can be accomplished by employing consecutive reactors for the production of methanol and subsequent conversion of methanol to dimethyl ether. For example, a synthesis gas-containing feedstock can be contacted in a synthesis gas-to-methanol production reactor with a synthesis gas-to-methanol conversion catalyst and at reaction conditions effective to convert at least a portion of the synthesis gas-containing feedstock to a product stream comprising methanol. At least a portion of such product stream methanol can be subsequently be contacted in a methanol conversion reactor with a methanol-to-dimethyl ether conversion catalyst and at reaction conditions effective to convert at least a first portion of the product stream methanol to dimethyl ether, forming the synthesis gas conversion reactor section effluent.

As will be appreciated by those skilled in the art and guided by the teaching herein provided, the reactors employed in such processing can desirably be tubular reactors with a circulating coolant, such as water, on the shell side, or adiabatic reactors such as with internal quench, interstage cooling, cooling coils or the like.

A synthesis gas conversion reactor zone effluent stream 24 such as typically at least comprising methanol and usually also at least comprising dimethyl ether and water is withdrawn from the synthesis gas conversion reactor zone 22. Those skilled in the art and guided by the teachings herein provided will appreciate that the production of dimethyl ether alone or together with, as in a blend, with methanol can be advantageous for the synthesis gas conversion reactor zone as the conversion of synthesis gas to dimethyl ether generally does not suffer the severe equilibrium limitations commonly encountered or associated with the primary conversion of synthesis gas to methanol. For example, the per pass conversion rate of synthesis gas can desirably be increased from about in the range of 30-40%, in the case of conversion of synthesis gas to methanol, to in the range of about 70-80% or higher in the case of conversion of synthesis gas to dimethyl ether. As a result, the size of equipment, such as the size of necessary process vessels, recycle compressors and the like, as well necessary energy inputs such as energy required for recycle compressor operation can dramatically be reduced.

The effluent stream 24, such as after cooling such as via one or more heat exchangers (not shown) is passed to a separation zone, generally designated by the reference numeral 26. The separation zone 26 may desirably include one or more separation sections such as each composed of one or more separation vessels, such as generally composed of one or more fractionation columns such that the various components can be appropriately separated, for example, such as a result of their different relative volatilities. In accordance with one embodiment, one such simple fractionation train may involve a first flash section in which noncondensible light ends like unconverted synthesis gas components are separated, followed by stripper or distillation column wherein dimethyl ether may be recovered overhead, and followed by another distillation column in which methanol is recovered overhead while water and heavier components (e.g., heavier alcohols and aldehydes) are rejected in the bottom. Those skilled in the art and guided by the teachings herein provided will appreciate the specific or particular sequencing of such separation steps can be appropriately altered and modified as required by the process conditions and economics. For example, whenever a distillation column is used, the operating conditions can desirably be chosen to entail a pressure sufficiently high so that the overhead vapors can be condensed by using either air cooling or cooling water, thus obviating the need for costlier refrigerated overhead condensation schemes. Thus such considerations will typically influence or dictate processing conditions such as the overall pressure requirements of the process cascade.

Through or as a result of such separation processing of the effluent stream 24, there is produced or formed a stream 30 such as generally composed of oxygenate materials such as methanol, dimethyl ether or a combination thereof, such as produced or formed by or in the synthesis gas conversion reactor zone 22.

Such separation processing also produces or forms a stream 32 such as a generally composed of water, and such as may additionally contain small amounts of other reaction species such as heavy impurities or by-products (e.g., heavy alcohols, aldehydes, etc.). Such a stream can be further treated for the removal of such heavy impurities and by-products and the water can, if desired, be recycled to the synthesis gas generation unit or, alternatively utilized such as in irrigation or other agricultural applications.

Such separation processing also produces or forms a stream 34 such as composed of at least a portion of the unreacted synthesis gas remaining in the synthesis gas conversion reactor zone effluent stream 24. As shown, such stream or selected portion thereof can desirably be recycled to the synthesis gas conversion reactor zone 22 for reaction processing such as to form or produce additional synthesis gas conversion reaction products.

Such separation processing may also produce or form, as shown, a stream 35 such as generally composed of methanol. The possible further desirable use of such a methanol stream is described in greater detail below.

Those skilled in the art and guided by the teachings herein provided will appreciate that various suitable separation zone arrangements can desirably be used in the practice of such embodiments. For example, a separation zone arrangement in accordance with one preferred embodiment desirably includes: a first separator for separating a vapor phase comprising unconverted synthesis gas and dimethyl ether from a condensate phase comprising liquid methanol and dimethyl ether; an absorber for absorbing dimethyl ether from the vapor phase using methanol and to form a first absorber process stream comprising unconverted synthesis gas and a second absorber process stream comprising dimethyl ether in methanol; and a second separator effective to separate dimethyl ether and methanol from each other in the second absorber process stream.

It is be understood, however, that the broader practice of the invention is not necessarily limited to or by specific or particular separation zone arrangements.

The oxygenate-containing stream 30 is passed via the line 36 and introduced into an oxygenate conversion reactor zone 40 wherein such oxygenate-containing feedstock materials contact with an oxygenate conversion catalyst at reaction conditions effective to convert the oxygenate-containing feedstock to form an oxygenate conversion effluent stream comprising fuel gas hydrocarbons, light olefins, and C₄ plus hydrocarbons, in a manner as is known in the art, such as, for example, utilizing a fluidized bed reactor.

In accordance with certain selected preferred embodiments, the oxygenate material stream 30, respectively alternatively, comprises, consists essentially of, or consists of methanol. For example, whereas crude methanol may typically contain 20 wt-% or more of water, in instances where the feed to a corresponding or associated oxygenate conversion reactor zone is required to be shipped or transported over relatively long distances, a higher grade methanol (e.g., methanol with a lesser water content) may be desired to reduce or minimize the costs associated with such shipping and transportation. In such cases, water may desirably be removed to produce methanol of at least 95 wt-% or better purity and, in accordance with certain embodiments, methanol at least 98 wt-% or better purity. A typical chemical-grade specification of “pure” methanol is 99.85 wt-%.

In accordance with certain other selected preferred embodiments, the oxygenate material stream 30, respectively alternatively, comprises, consists essentially of, or consists of dimethyl ether. For example, equilibrium constraints generally dictate that the production of dimethyl ether from methanol on a once-through basis (e.g., without methanol separation and recycling to the dimethyl ether production section), the product will generally comprise about 80 wt-% dimethyl ether and a balance of methanol, on a water-free basis.

Through the use of feeds of dimethyl ether, either alone (i.e., without significant relative amounts of other oxygenates such as methanol, etc.) or in combination with methanol the oxygenate conversion process can be significantly enhanced such as by significantly reducing required volumetric flow rates and thus, the size of required processing vessels.

For example, in the production of ethylene and propylene at about 1:1 weight ratio (1 mol of ethylene and ⅔ mol of propylene) from methanol, the production of 1 mol of ethylene and ⅔ mol of propylene from methanol will also produce 4 mols of water. On the other hand, if the same amount of ethylene and propylene is produced from dimethyl ether, only 2 mols of water are coproduced. Therefore, the total number of mols in the effluent from the reactor (relative to one mol of ethylene) is reduced from 5 ⅔ to 3 ⅔ or about a 35% reduction. Such reduction in the number of mols represents an equivalent reduction in the volumetric flow rate of effluent from the reactor, and thus a smaller reactor vessel and downstream processing equipment.

In addition to beneficially reducing the size of required reactor, the use of dimethyl ether rather than methanol as the feed to such an oxygenate conversion reactor can also beneficially result in less heat release by the process. Consequently, there can be additional savings such as associated with reduced heat exchange surface or elimination of catalyst coolers, for example.

Still further, because the above-identified reduction in mols is accomplished by reducing the mols of water, the partial pressure of water is reduced, thus improving the stability of the catalyst used in the conversion of oxygenates to light olefins.

In accordance with certain embodiments, the oxygenate-containing feedstock desirably comprises about 10 to about 30 mol-% methanol and about 70 to about 90 mol-% dimethyl ether.

Reaction conditions for the conversion of oxygenates to light olefins are known to those skilled in the art. Preferably, in accordance with particular embodiments, reaction conditions comprise a temperature between about 200° and about 700° C., more preferably between about 300° and 600° C., and most preferably between about 400° and about 550° C. As will be appreciated by those skilled in the art and guided by the teachings herein provided, the reactions conditions are generally variable such as dependent on the desired products. For example, if increased ethylene production is desired, then operation at a reactor temperature between about 475° and about 550° C. and more preferably between about 500° and about 520° C., may be preferred. If increased propylene production is desired, then operation at a reactor temperature between about 350° and about 475° C. and more preferably between about 400° and about 430° C. may be preferred. The light olefins produced can have a ratio of ethylene to propylene of between about 0.5 and about 2.0 and preferably between about 0.75 and about 1.25. If a higher ratio of ethylene to propylene is desired, then the reaction temperature is generally desirably higher than if a lower ratio of ethylene to propylene is desired.

The oxygenate conversion reactor zone 40 produces or results in an oxygenate conversion reactor zone effluent stream 42 such as generally comprising fuel gas hydrocarbons, by-product dimethyl ether, light olefins, and C₄ plus hydrocarbons, as well as possible some carbon oxides (e.g., CO and C0₂).

In accordance with one preferred embodiment, at least a portion of such dimethyl ether can be separated and recovered from the oxygenate conversion reactor zone effluent stream 42 and in turn, recycled to the oxygenate conversion reactor zone 40 for reaction. Thus, as shown in the FIGURE, the oxygenate conversion reactor zone effluent stream 42 is introduced into a dimethyl ether recovery zone 46 such as in the form of at least one absorber such as desirably employs methanol to absorb by-product dimethyl ether from the oxygenate conversion reactor zone effluent stream 42. In accordance with one preferred embodiment, at least a portion of the methanol required to realize the desired dimethyl ether absorption is supplied by or as a result of the introduction into the dimethyl ether recovery zone 46 of at least a portion of the above-described stream 35 generally composed of methanol such as via the line 48.

As an alternative or as a supplement to such process generated methanol, required or desired methanol can be provided or supplied from some alternate source or supply such as signified by the stream 49 and via the line 48.

As a further alternate or supplement to such methanol use, water (such as exemplarily introduced via the line 51) can be used to absorb dimethyl ether.

As a result of such absorption of by-product dimethyl ether, a stream 50 such as generally containing at least dimethyl ether is formed. In accordance with one embodiment, in addition to such by-product dimethyl ether, the stream 50 additionally contains or includes at least a portion of methanol and/or water in which the dimethyl ether has been absorbed. Alternatively, if desired, at least a portion of the absorbed dimethyl ether can be separated from the methanol and/or water in a first separator. Further, in accordance with one embodiment, at least a portion of such separated dimethyl ether can subsequently be fed to the oxygenate conversion reactor zone for reaction processing. If desired, at least a portion of any such separated methanol and/or water can be recycled and used for further recovery of dimethyl ether.

The stream 50 with or without further processing can, if desired, be introduced into the oxygenate conversion reactor zone 40, such as via the line 36, for further oxygenate conversion processing.

The dimethyl ether recovery zone 46 may also result in the formation of a stream 54 such as generally constituting the remaining portion of the oxygenate conversion reactor zone effluent after such dimethyl ether recovery zone treatment. As shown, the stream 54 may be passed to a product separation and recovery zone 60, such as known in the art, for the appropriate desired product separation and recovery. For example, in accordance with one preferred embodiment, a suitable such product separation and recovery zone may comprise an appropriate gas concentration system.

Gas concentration systems, such as used in the processing of the products resulting from such oxygenate conversion processing, are well known to those skilled in the art and do not generally form limitations on the broader practice of the invention as those skilled in the art and guided by the teachings herein provided will appreciate.

In the product separation and recovery zone 60, the remaining portion of the oxygenate conversion reactor zone effluent may desirably be processed such as to provide a fuel gas stream 62, an ethylene stream 64, a propylene stream 66 and a mixed C₄ plus hydrocarbon stream 70, such as generally composed of butylene and heavier hydrocarbons. In order to facilitate illustration and discussion, those skilled in the art and guided by the teachings herein provided will appreciate that other additional or alternative products streams such as may be formed from the oxygenate conversion product stream via such a product separation and recovery zone have not here been shown or are here described in great detail.

While the invention has been described above making specific references to embodiments wherein methanol employed for the recovery and desirable recycle of at least a portion of the dimethyl ether remaining after oxygenate conversion processing is internally generated via synthesis gas conversion, those skilled in the art and guided by the teachings herein provided will appreciate that the broader practice of the invention is not necessarily so limited. For example, if desired or if preferred, a suitable processing scheme in accordance with another embodiment may employ methanol supplied or provided by a selected alternative methanol source or supply.

Embodiments, such as described above, incorporating and utilizing synthesis gas conversion to form an effluent including product dimethyl ether, subsequent separation of such product dimethyl ether and conversion thereof to form light olefins desirably provides or results in improved processing such as by minimizing or at least reducing the size of required vessels.

The invention illustratively disclosed herein suitably may be practiced in the absence of any element, part, step, component, or ingredient which is not specifically disclosed herein.

While in the foregoing detailed description this invention has been described in relation to certain preferred embodiments thereof, and many details have been set forth for purposes of illustration, it will be apparent to those skilled in the art that the invention is susceptible to additional embodiments and that certain of the details described herein can be varied considerably without departing from the basic principles of the invention.

What is claimed is:

1. An integrated process for oxygenate synthesis and conversion to light olefins, said process comprising:
contacting a synthesis gas-containing feedstock in a synthesis gas conversion reactor zone with a synthesis gas conversion catalyst material and at reaction conditions effective to produce a synthesis gas conversion reactor zone effluent comprising at least methanol;
contacting an oxygenate-containing feedstock comprising at least one oxygenate-containing feedstock material selected from the group consisting of methanol and dimethyl ether in an oxygenate conversion reactor zone with an oxygenate conversion catalyst and at reaction conditions effective to convert the oxygenate-containing feedstock to produce an oxygenate conversion reactor zone effluent comprising light olefins and dimethyl ether; and
contacting at least a portion of the oxygenate conversion reactor zone effluent with at least a portion of the synthesis gas conversion reactor zone effluent methanol effective to recover dimethyl ether from the oxygenate conversion reactor zone effluent.

2. The process of claim 1 additionally comprising introducing dimethyl ether recovered from the oxygenate conversion reactor zone effluent into the oxygenate conversion reactor zone for contact with the oxygenate conversion catalyst at reaction conditions effective to convert at least a portion of the dimethyl ether recovered from the oxygenate conversion reactor zone effluent to light olefins.

3. The process of claim 1 wherein the oxygenate-containing feedstock comprises methanol.

4. The process of claim 3 wherein the oxygenate-containing feedstock additionally comprises dimethyl ether.

5. The process of claim 1 wherein the oxygenate-containing feedstock comprises dimethyl ether.

6. The process of claim 1 wherein the contacting of the synthesis gas-containing feedstock in the synthesis gas conversion reactor zone comprises contacting the synthesis gas-containing feedstock in a synthesis gas conversion reactor zone with a synthesis gas conversion catalyst material and at reaction conditions effective to produce the synthesis gas conversion reactor zone effluent, wherein the synthesis gas conversion reactor zone effluent additionally comprises product dimethyl ether, other synthesis gas conversion products and unreacted synthesis gas, the other synthesis gas conversion products comprising the methanol and water; and wherein the process additionally comprises separating unreacted synthesis gas from the product dimethyl ether and the other synthesis gas conversion products; and separating methanol from the product dimethyl ether and the other synthesis gas conversion products to form the synthesis gas conversion reactor zone effluent methanol.

7. The process of claim 1 wherein said contacting of at least a portion of the oxygenate conversion reactor zone effluent with at least a portion of the synthesis gas conversion reactor zone effluent methanol effective to recover dimethyl ether from the oxygenate conversion reactor zone effluent comprises the synthesis gas conversion reactor zone effluent methanol is effective to absorb dimethyl ether from the oxygenate conversion reactor zone effluent and wherein the process additionally comprises separating at least a portion of the absorbed dimethyl ether from the methanol in a first separator; and feeding at least a portion of the separated dimethyl ether to the oxygenate conversion reactor zone.

8. An integrated process for oxygenate synthesis and conversion to light olefins, said process comprising:
contacting a synthesis gas-containing feedstock in a synthesis gas conversion reactor zone with a synthesis gas conversion catalyst material and at reaction conditions effective to produce a synthesis gas conversion reactor zone effluent comprising product dimethyl ether, other synthesis gas conversion products and unreacted synthesis gas, the other synthesis gas conversion products comprising methanol and water;
separating unreacted synthesis gas from the product dimethyl ether and the other synthesis gas conversion products;
recycling the separated unreacted synthesis gas to the synthesis gas conversion reactor zone for contact with the catalyst material at reaction conditions effective to produce additional synthesis gas conversion reactor zone effluent;
separating at least a portion of the other synthesis gas conversion product methanol from the product dimethyl ether and from the other synthesis gas conversion product water;
contacting an oxygenate-containing feedstock comprising methanol and dimethyl ether in an oxygenate conversion reactor zone with an oxygenate conversion catalyst and at reaction conditions effective to convert the oxygenate-containing feedstock to produce an oxygenate conversion reactor zone effluent comprising light olefins and dimethyl ether;
contacting at least a portion of the oxygenate conversion reactor zone effluent with at least a portion of the separated other synthesis gas conversion reactor zone effluent methanol effective to recover dimethyl ether from the oxygenate conversion product stream; and
recycling the recovered dimethyl ether to the oxygenate conversion reactor zone for contact with the oxygenate conversion catalyst at reaction conditions effective to convert the oxygenate-containing feedstock to produce additional oxygenate conversion reactor zone effluent.

9. The process of claim 8 additionally comprising separating at least a portion of the recovered dimethyl ether from the methanol used to effect such recovery.

10. The process of claim 8 wherein the oxygenate-containing feedstock comprises about 10 to about 30 mol-% methanol and about 70 to about 90 mol-% dimethyl ether.

11. An integrated system for oxygenate synthesis and conversion to light olefins, said system comprising:
a synthesis gas conversion reactor zone for contacting a synthesis gas-containing feedstock with a synthesis gas conversion catalyst and at reaction conditions effective to convert the synthesis gas-containing feedstock to produce a synthesis gas conversion reactor zone effluent comprising product dimethyl ether, other synthesis gas conversion products and unreacted synthesis gas, the other synthesis gas conversion products comprising methanol and water;
a separation zone effective for separating the synthesis gas conversion reactor zone effluent to form a recycle stream of unconverted synthesis gas, a first process stream comprising methanol and an oxygenate-containing feed stream comprising at least one oxygenate-containing material selected from the group consisting of methanol and dimethyl ether;
an oxygenate conversion reactor zone for contacting at least a portion of the oxygenate-containing feed stream with an oxygenate conversion catalyst and at reaction conditions effective to convert the oxygenate-containing feed stream to produce an oxygenate conversion reactor zone effluent comprising light olefins and dimethyl ether; and
a separation system including effective to separate dimethyl ether from the oxygenate conversion reactor zone effluent via methanol absorption of such dimethyl ether.

12. The system of claim 11 wherein oxygenate-containing feed stream comprises a combination of methanol and dimethyl ether.

13. The system of claim 11 wherein the separation zone comprises a first separator for separating a vapor phase comprising unconverted synthesis gas and dimethyl ether from a condensate phase comprising liquid methanol and dimethyl ether; an absorber for absorbing dimethyl ether from the vapor phase using methanol and to form a first absorber process stream comprising unconverted synthesis gas and a second absorber process stream comprising dimethyl ether in methanol; and a second separator effective to separate dimethyl ether and methanol from each other in the second absorber process stream.

14. A method for producing light olefins, said method comprising:
contacting an oxygenate-containing feedstock comprising at least one oxygenate-containing feedstock material selected from the group consisting of methanol and dimethyl ether in an oxygenate conversion reactor with an oxygenate conversion catalyst and at reaction conditions effective to convert the oxygenate-containing feedstock to produce an oxygenate conversion reactor effluent comprising light olefins and dimethyl ether;
contacting at least a portion of the oxygenate conversion reactor effluent with a quantity of methanol to absorb at least a portion of the dimethyl ether from the oxygenate conversion reactor effluent;
separating at least a portion of the absorbed dimethyl ether from the methanol in a first separator; and
feeding at least a portion of the separated dimethyl ether to the oxygenate conversion reactor.

15. The method of claim 14 additionally comprising returning at least a portion of the separated methanol to absorb dimethyl ether from the oxygenate conversion reactor effluent.

16. The method of claim 14 wherein the oxygenate-containing feedstock comprises dimethyl ether.

17. The method of claim 16 wherein the oxygenate-containing feedstock additionally comprises methanol.

18. The method of claim 17 wherein the oxygenate-containing feedstock comprises about 10 to about 30 mol-% methanol and about 70 to about 90 mol-% dimethyl ether.

19. The method of claim 14 wherein the oxygenate-containing feedstock is formed by a process comprising contacting a synthesis gas-containing feedstock in a synthesis gas conversion reactor zone with a synthesis gas conversion catalyst material and at reaction conditions effective to produce a synthesis gas conversion reactor zone effluent comprising at least methanol, and treating the synthesis gas conversion reactor zone effluent to form the oxygenate-containing feedstock.
